(19) United States
(12) Patent Application Publication
(10) Pub. No.: US 2006/0008875 A1
(43) Pub. Date: Jan. 12, 2006

(54) Novel TFIIH subunit

(75) Inventors: Giuseppina Giglia-Mari, Rotterdam (NL); Deborah Hoogstraten, Rotterdam (NL); Arjan Theil, Delfgauw (NL); Nils Wijgers, Papendrecht (NL); Nicolaas G.J. Jaspers, Rotterdam (NL); Anja Raams, Rotterdam (NL); Jan H.J. Hoeijmakers, Moerkapelle (NL); Wim Vermeulen, Zwijndrecht (NL)

Correspondence Address: THE WEBB LAW FIRM, P.C., 700 KOPPERS BUILDING, 436 SEVENTH AVENUE, PITTSBURGH, PA, 15219, US

(73) Assignee: Universitair Medisch Centrum Rotterdam, Rotterdam (NL)

(21) Appl. No.: 10/885,510

(22) Filed: Jul. 6, 2004

(51) Int. Cl. C07H 21/04; C12P 21/06; C07K 14/82

(52) U.S. Cl. 435/69.1; 435/320.1; 435/325; 530/350; 536/23.5

(57) ABSTRACT

The present invention pertains to a nucleic acid sequence encoding a human TFIIH 8 kDa subunit and related sequences. The nucleic acids may be used in methods for producing a TFIIH subunit, as well as in methods for diagnosing or treating transcription and NER deficiencies, in particular in some forms of trichothiodystrophy (TTD). The hTFB5/TTDA gene and encoded protein may be used for therapy or genetherapy products, aimed at treating congenital NER disorders and may also be used in methods of diagnosis of disorders in basal transcription, NER and TCR activity in mammals, using molecular probes or antibodies specific for TTDA.

### FIELD OF THE INVENTION

The present invention relates to the fields of cell biology and medicine, in particular to gene transcription and DNA repair systems, more in particular to the detection and treatment of defects in basal and activated transcription, nucleotide excision repair (NER) and transcription coupled repair systems in mammals and in mammalian cells.

### BACKGROUND OF THE INVENTION

TFIIH is a multicomponent basal transcription factor complex. Transcription factor II H interacts with a variety of factors during transcription, including nuclear receptors, tissue-specific transcription factors, chromatin remodeling complexes and RNA, suggesting that, in addition to its essential role in transcription initiation for RNA polymerases I and II, it also participates as a regulatory factor. To date, at least nine subunits have been identified within the TFIIH holoenzyme complex in mammals. Various enzymatic activities, including DNA repair, helicase, and cyclin dependent kinase activities, have been reported. The nine known subunits of the TFIIH complex are summarized in table 2.

TFIIH is involved in transcription from RNA polymerase I (ribosomal RNA's) and RNA polymerase II (messenger RNA) transcription. TFIIH is essential for promoter melting, the separation of the DNA strands that make up the double helix, and/or for promoter clearance (i.e. for pol II to break free of the initiation complex into elongation mode). Surprisingly, TFIIH also is essential for the core reaction of Nucleotide Excision Repair (NER) of damaged DNA templates. In addition, TFIIH appears to play an important role in a subpathway of NER, the preferential repair of actively transcribed regions on the DNA; the so-called transcription coupled DNA repair (TCR) process which is restricted to the repair of NER-type of lesions but also includes the TCR of oxidative DNA lesions.

The XPB, XPD, p62, p52, p44, and p34 subunits are thought to constitute the “core” of the TFIIH transcription machinery. The two largest TFIIH subunits, XPB and XPD, are ATP-dependent helicases of opposite polarity, crucial for promoter melting. Two of the smaller subunits have possible zinc finger domains. Although the p44 and p34 subunits have no defined enzymatic activity, their zinc finger structures suggest that they may be DNA-binding proteins that might mediate interactions with soluble transcription factors. TFIIH appears to be dependent upon TFIIE for incorporation into the pre-initiation complex, the assembly of basal transcription factors and the polymerase on promoters of transcribed genes. The Cdk-activating kinase (CAK) subcomplex, comprising subunits Cdk7, cyclin H, and MAT1, phosphorylate several cyclin-dependent kinases (Cdks). The associated kinase activity (also referred to as TFIIK) can phosphorylate the C-terminal domain of the RNA pol II largest subunit. This phosphorylation at serines and threonines is essential for DNA transcription, i.e. for elongation of the RNA chain.

Besides the central role TFIIH plays in RNA polymerase I and II transcription, it is also involved in nucleotide excision repair (NER) and transcription-coupled repair (TCR) (Egly, J. M. FEBS Lett. 498, 124-128 (2001). NER removes a wide range of DNA helix-distorting injuries, including ultraviolet light (UV)-induced lesions (Hoeijmakers, J. H., Nature 411, 366-374, 2001). TCR is a repair pathway that eliminates both classical NER lesions and oxidative DNA injuries from the transcribed strand (de Boer, J. et al., Science 296, 1276-1279, 2002 and Le Page, F. et al., Cell 101, 159-171, 2000).

Several hereditary mutations in repair/transcription factor TFIIH subunits are associated with three photo-hypersensitive syndromes in man: 1) xeroderma pigmentosum (XP), 2) XP combined Cockayne syndrome (CS), a neuro-developmental disorder, and 3) the CS-like brittle hair disease trichothiodystrohpy (TTD) (Lehmann, A. R., Genes Dev 15, 15-23, 2001 and Bootsma et al., in Nucleotide Excision Repair Syndromes: Xeroderma Pigmentosum, Cockayne Syndromes, And Trichothiodystrophy., 245-274, New york, 1998 ), summarized in table 1. Photosensitive features of XP patients are mainly caused by defective NER, whereas the CS and TTD features are attributed to an affected TCR and transcription function, which result in premature or enhanced ageing symptoms or phenotypes.

Trichothiodystrophy (TTD) is a rare autosomal recessive disorder characterized by sulfur-deficient brittle hair and ichthyosis (Itin et al. 2001). Hair shafts split longitudinally into small fibers, and this brittleness is associated with levels of cysteine/cystine in hair proteins that are 15 to 50% of those in normal individuals. The hair has characteristic “tiger-tail” banding visible under polarized light. The patients often have an unusual facial appearance, with protruding ears and a receding chin. Mental abilities range from low normal to severe retardation. Several categories of the disease can be recognized on the basis of cellular responses to UV damage and the affected gene.

Evidence from complementation tests by cell fusion experiments identified that the TTD phenotype can be caused by mutations in at least 2 separate genes: ERCC2/XPD and ERCC3/XPB, which encode the 2 helicase subunits of transcription/repair factor TFIIH (Broughton, B. C. et al., Hum. Mol. Genet. 10, 2539-2547, 2001, Botta, E. et al., Am. J. Hum. Genet. 63, 1036-1048, 1998, Weeda, G. et al. Am. J. Hum. Genet. 60, 320-329, 1997, Graham, J. M., Jr. et al. Am. J. Hum. Genet. 69, 291-300, 2001). Besides XP groups B and D, an exceptional trichothiodystrophy complementation group designated TTD-A was identified by Stefanini et al., (Am J Hum Genet 53, 817-21, 1993). The third, yet unidentified gene causing photosensitive TTD (Vermeulen, W. et al., Cold Spring Harb. Symp. Quant. Biol. 59, 317-329, 1994), designated TTDA also appeared associated with TFIIH (Vermeulen, W. et al., Nat. Genet. 26, 307-13, 2000). Table 1 summarizes the clinical and cellular features of all known TTD-A cases, including two new group A families. Cells from TTD-A patients are only mildly UV-sensitive despite the low UV-induced DNA repair synthesis (Unscheduled DNA Synthesis (UDS), non-replicative DNA synthesis which takes place throughout the entire cell cycle, including G1-, S-, and G2-phase of the cell cycle). Remarkably, in the third form of DNA-repair deficient TTD complementation group A, none of the nine TFIIH encoding subunits carried mutations; instead the steady-state level of the entire complex was severely reduced. A comprehensive and frequently updated list of 363 TTD, CS and XP mutations in humans can be found on www.xpmutations.org.

Previously, the inventors found that highly purified TFIIH corrected the NER defect of TTD-A cells, although none of the nine TFIIH genes were mutated in TTD-A. Moreover, TFIIH isolated from TTD-A cells displayed normal in vitro enzymatic activities and transiently restored the NER defect in TTD-A cells after microinjection, suggesting that TFIIH was qualitatively not or only mildly affected.

The current invention provides for nucleic acids that encode a novel 8 kDa subunit of TFIIH and that may be used in the diagnosis and therapy of the nucleotide excision repair (NER) deficiencies, transcription coupled repair (TCR) deficiencies and DNA transcription deficiencies, in particular trichothiodistrophy.

### DETAILED DESCRIPTION OF THE INVENTION

Here we describe the identification of the human TFB5 ortholog and its association with human TFIIH. Recently, a novel, tenth TFIIH subunit (TFB5), was identified in yeast. Within a quantitative proteomic screening of promoter-associated RNA polymerase II preinitiation complexes from yeast nuclear extracts, a novel, very small protein of 72 amino acids (˜8 kDa) was identified and shown to be a core component of yeast TFIIH, designated TFB5 (Ranish, J. A. et al., Nat. Genet. 2004). This protein was overlooked in previous analyses of TFIIH due to its exceptionally small size.

Database screening identified several orthologs of TFB5 (FIG. 5), including a presumed human homologue. The human TFB5 gene codes for a 71 amino acid polypeptide with a predicted molecular weight of 8 kDa (referred to as p8). TFB5 appeared highly conserved, with a sequence identity of 25% and 56% similarity between human and yeast. The strong evolutionary conservation of each TFIIH subunit (Table 2 and FIG. 5) in combination with the overall structural homology of the complex (Schultz, P. et al., Cell 102, 599-607, 2000 and Chang, W. H. & Kornberg, R. D., Cell 102, 609-613, 2000), prompted us to investigate whether human TFB5 is also part of mammalian TFIIH and whether it is the enigmatic TTDA factor.

Microinjection of hTFB5 cDNA corrected the DNA repair defect of TTD-A cells and three functional inactivating mutations were identified in three unrelated TTD-A families. This TTDA gene product was shown to play a role in regulating the level of TFIIH and significantly stabilizes the TFIIH complex in vivo. The identification of a new evolutionarily conserved subunit of TFIIH implicated in TTD-A, provides important solutions for diagnosing and treating TFIIH dysfunction in transcription, DNA repair systems, in particular NER and TCR systems and human diseases associated with NER and TCR dysfunction.

In a first aspect the invention relates to a nucleic acid molecule comprising a sequence encoding a polypeptide with TFIIH stabilizing activity and/or NER promoting activity. The nucleic acid molecule is preferably selected from the group consisting of: (a) nucleic acid molecules encoding a polypeptide comprising an amino acid sequence having at least 50, 60, 70, 80, 90, 95, 98 or 99% identity with the amino acid sequence of SEQ ID NO:2 ; (b) nucleic acid molecules the complementary strand of which hybridises to a nucleic acid molecule having a nucleotide sequence as depicted in SEQ ID NO:2 ; and, (c) nucleic acid molecules the sequence of which differs from the nucleic acid molecule of (a) or (b) due to the degeneracy of the genetic code.

A polypeptide with TFIIH stabilizing and/or NER promoting activity is herein understood to mean a polypeptide that is capable of stabilizing TFIIH as determined in a functional assay, preferably the assay herein provided in example 4, and/or active in NER promoting activity as determined by a functional NER assay and/or an assay specific for the TCR subpathway, preferably the assay provided in example 3. A further functional assay for TFIIH stabilizing and/or NER promoting activity is provided by complementation of TTD-A cells (i.e. TTD cells that belong to the TTD-A complementation group). A polypeptide with TFIIH stabilizing and/or NER promoting is herein understood to be capable of correcting the DNA repair defect of TTD-A cells, e.g. by microinjection of the polypeptide or a nucleic acid construct capable of expressing the polypeptide.

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartic acid and glutamic acid and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gin or glu; Met to leu or ile; Phe to met, leu or tyr; Ser to thr; Thr to ser; Trp to tyr; Tyr to trp or phe; and, Val to ile or leu.

Nucleic acid sequences encoding polypeptides having TFIIH stabilizing activity and/or NER promoting activity may also be defined by their capability to hybridise with the (complementary strand of) the nucleotide sequence of SEQ ID NO: 2, preferably under moderate, or more preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 99%.

The nucleic acid molecules of the invention preferably encode a polypeptide with TFIIH stabilizing and/or NER promoting activity that is a mammalian, preferably a human TTDA protein or a murine TTDA protein.

In a further aspect, the invention relates to a nucleic acid probe comprising a part of a nucleotide sequence that has at least 90% with a nucleotide sequence as depicted in SEQ ID NO: 2, or its complement sequence. Preferably, the probe comprises a part of a nucleotide sequence that has at least 90% with a nucleotide sequence as depicted in SEQ ID NO: 2 from positions 1 to 331 more preferably from positions 60 to 275. Nucleic acid probes are used to analyse nucleic acids encoding the polypeptide having TFIIH stabilizing and NER promoting activity. Such analysis may include the analysis of the genotype of a subject with respect to sequence encoding TTDA, e.g. the presence or absence of specific mutations or alleles, or may involve analysis of the expression of nucleic acids encoding TTDA. The subject may be a mammal and preferably is a human. Such analyses may be performed for diagnostic purposes (see below). Nucleic acid probes thus include poly- and oligonucleotides for use as hybridisation probes, as primers for sequencing or amplification and for use in techniques such as Oligonucleotide Ligation (-Amplification) assays (see e.g. U.S. Pat. No. 4,988,617; U.S. Pat. No. 5,876,924; WO 96/15271; and WO 97/45559) or Single-Strand Conformational Polymorphism assays (see e.g. Orita et al., 1989, Genomics 5: 874-879). For further nucleic acid analysis techniques reference is made to Sambrook and Russell (2001, Molecular Cloning: A Laboratory Manual, 3^{rd} edition, Cold Spring Harbor Laboratory Press, Cold Spring Harbor N.Y.). A preferred nucleic acid probe is a probe that comprises at least 10, 12, 14, 16 or 20 contiguous nucleotides of a nucleotide sequence that has at least 70, 80 or 90% identity with a nucleotide sequence as depicted in SEQ ID NO: 2, which spans the entire coding sequence of the human hTFB5/TTDA gene. Probes derived from SEQ ID NO: 2 or the genomic TTDA locus, SEQ ID. NO. 3, 4 and 5, may thus be used to analyse both exonic and intronic sequences as well as non-coding sequence up- or downstream from the transcription unit that may be involved in the regulations of expression of the TTDA/hTFB5 gene. A probe according to the current invention comprising a stretch of nucleic acids may be advantageously physically linked to a solid support, such as, but not limited to, a DNA (micro-)array.

In another aspect the invention relates to a vector comprising a nucleic acid molecule or probe as defined above. Preferably the vector is a replicative vector comprising an origin of replication (or autonomously replication sequence) that ensures multiplication of the vector in a suitable host for the vector. Alternatively the vector is capable of integrating into the host cell's genome, e.g. through homologous recombination, random integration or otherwise.

A particularly preferred vector is an expression vector that wherein a nucleotide sequence encoding a polypeptide with TFIIH stabilizing and/or NER promoting activity as defined above, is operably linked to a promoter capable of directing expression of the coding sequence in a host cell for the vector.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues.

Expression vectors allow the polypeptides with TFIIH stabilizing and/or NER promoting activity of the present invention to be prepared using recombinant techniques in which a nucleotide sequence encoding the polypeptide of interest is expressed in suitable cells, e.g. cultured cells or cells of a multicellular organism, such as described in Ausubel ed., “Current Protocols in Molecular Biology”, Greene Publishing and Wiley-Interscience, New York (2003) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

Typically, nucleic acids encoding the desired polypeptides are used in expression vectors. The phrase “expression vector” generally refers to nucleotide sequences that are capable of affecting expression of a gene in hosts compatible with such sequences. These expression vectors typically include at least suitable promoter sequences and optionally, transcription termination signals. Additional factors necessary or helpful in effecting expression can also be used as described herein. DNA encoding a polypeptide is incorporated into DNA constructs capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines.

DNA constructs prepared for introduction into a particular host typically include a replication system recognised by the host, the intended DNA segment encoding the desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof. The selection of an appropriate promoter sequence generally depends upon the host cell selected for the expression of the DNA segment. Examples of suitable promoter sequences include prokaryotic, and eukaryotic promoters well known in the art (see, e.g. Sambrook and Russell, 2001, supra). The transcriptional regulatory sequences typically include a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S.cerevisiae, insect cells, e.g., Sf9 cells, mammalian cells, e.g., CHO cells and bacterial cells, e.g., E. coli.

In vitro mutagenesis and expression of mutant proteins are described generally in Ausubel et al. (2003, supra) and in Sambrook and Russell (2001, supra). Also see, Kunkel (1985, supra; describing site directed mutagenesis) and Roberts et al. (1987, supra; describing cassette mutagenesis).

Another method for preparing polypeptides is to employ an in vitro transcription/translation system. DNA encoding a polypeptide is cloned into an expression vector as described supra. The expression vector is then transcribed and translated in vitro. The translation product can be used directly or first purified. Polypeptides resulting from in vitro translation typically do not contain the post-translation modifications present on polypeptides synthesised in vivo, although due to the inherent presence of microsomes some post-translational modification may occur. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987.

In a further aspect the invention thus relates to a host comprising a vector as defined above. The host cells may be prokaryotic or eukarotic host cells as indicated above. The host cell may be a host cell that is suitable for culture in liquid or on solid media. Alternatively, the host cell is a cell that is part of a multicellular organism such as a transgenic plant or animal, preferably a non-human animal.

A further aspect the invention relates to a method for producing a polypeptide with TFIIH stabilizing activity and/or NER promoting activity. The method comprises the step of culturing a host cell as defined above under conditions conducive to the expression of the polypeptide. Optionally the method may comprise recovery the polypeptide. The polypeptide may e.g. be recovered from the culture medium by standard protein purification techniques, including a variety of chromatography methods known in the art per se.

Another aspect of the invention relates to a transgenic animal comprising in its somatic and germ cells a vector as defined above. The transgenic animal preferably is a non-human animal. Methods for generating transgenic animals are e.g. described in WO 01/57079 and in the references cited therein. Such transgenic animals may be used in a method for producing a polypeptide with TFIIH stabilizing activity and or NER promoting activity, the method comprising the step of recovering a body fluid or tissue from a transgenic animal comprising the vector or a female descendant thereof, wherein the body fluid contains the polypeptide, and, optionally recovery of the polypeptide from the body fluid. Such methods are also described in WO 01/57079 and in the references cited therein. The body fluid containing the polypeptide preferably is blood or more preferably milk. In another embodiment the transgenic animal may be a knock out animal, wherein the endogenous TTDA allele(s) have been replaced with a dysfunctional or mutated allele(s) via homologous recombination techniques known in the art. For instance the TTDA reading frame may be deleted or disrupted by insertion of a sequence and/or fused to selectable marker Neomycin R(esistance), Hygromycin R, DHFR, puromycin R, HSVtk or reporter genes such as luciferase. Also bi-cistronic messengers of TTDA and a marker allele maybe constructed using an IRES sequence. In other embodiments conditional knock outs or knock in animals may be constructed using the Cre-Lox or Frt/Flp recombinase systems known in the art. In yet another embodiment the transgenic animal may be a knock-in animal in which one or both copies of the TTDA alleles are replaced by sequences coding for tagged forms of TTDA. Various tags that are suitable for fusion with the TTDA reading frame, including e.g. fluorescent proteins such GFP and variants thereof, as well as affinity tags, are well known in the art (see below). In yet another embodiment RNA interference (RNAi) strategies known in the art and RNAi molecules may be applied to inactivate expression of TTDA locally or systemically in a mammal or in cells.

A further aspect of the invention relates to a polypeptide polypeptide with TFIIH stabilizing activity and or NER promoting activity and comprising an amino acid sequence having at least 80, 90, 95, 98 or 99% identity with the amino acid sequence of SEQ ID NO: 1. The polypeptide is further preferably as described above. Included within the scope of the invention are mutant polypeptides having TFIIH stabilizing activity and or NER promoting activity with e.g. altered specificity or additional features, such as but not limited to fusions to molecular tags that may be used for (but not limited to) purification/isolation, identification, visualization/localization or immunization purposes. The amino acid sequence of SEQ ID No. 1 may be fused to an HA, FLAG, myc, multiple-His, double HA, tubulin, GST, protein A, MBP, biotin or any other tag known in the art. Also fluorescent tags may be advantageously applied, such as GFP, EGFP, eCFP, eYFP, dsRed, RFP, GFP2, FLASH, SNAP and other fluorescent protein tags, or tags that bind specifically to fluorescent small molecular entities, quantum dots or any molecules known in the art. Based on the specific activities of TTDA in stabilizing TFIIH, promoting NER and TCR and promoting DNA transcription, the skilled person will know how to modify essential amino acids by minor amino acid substitutions and or deletions, insertion and fusions to alter its cellular activities, increase or decrease affinities for its natural or artificial binding partners in the TFIIH complex, increase the half-life of the protein, fuse it to tags or fluorescent reporters as mentioned above. The tertiary structure of a TTDA protein based on the enzyme depicted in SEQ ID NO: 1 may be altered applying the same principle as above to adapt the structure to modulate the structure of the interaction sites of the protein and subsequently assay the mutant for its TFIIH stabilizing activity and/or its promoting activity for NER, TCR and DNA transcription.

Another aspect of the invention relates to an antibody or antibody-fragment that specifically binds to a polypeptide with TFIIH stabilizing and/or NER promoting activity as defined above. Methods for generating monoclonal or polyclonal antibodies or antibody-fragments that specifically binds to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; U.S. Pat. No. 6,420,113 and WO02/085944 and references cited therein, and comprise V_{L}, V_{H}, scFv, Fab, Fab₂ fragments as well as camelid single chain antibodies or antibodies or antibody-fragments from phage-display libraries. The term “specific binding,” as used herein, includes both low and high affinity specific binding. Specific binding can be exhibited, e.g., by a low affinity antibody or antibody-fragment having a Kd of at least about 10-4 M. Specific binding also can be exhibited by a high affinity antibody or antibody-fragment, for example, an antibody or antibody-fragment having a Kd of at least about of 10-7 M, at least about 10-8 M, at least about 10-9 M, at least about 10-10 M, or can have a Kd of at least about 10-11 M or 10-12 M or greater. Antibodies may be raised in any mammal, and antibodies may preferably be humanized or deimmunized.

Another aspect of the invention relates to microarrays (or other high throughput screening devices) comprising the nucleic acids or polypeptides as defined above. A microarray is a solid support or carrier containing one or more immobilised nucleic acid or polypeptide fragments for analysing nucleic acid or amino acid sequences or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001, Curr. Opin. Chem. Biol. 5: 40-45). Microarrays comprising the nucleic acids may be applied e.g. in methods for analysing TTDA genotypes as indicated below. Microarrays comprising polypeptide may be used for detection of suitable candidates of TTDA mutants with altered affinities for binding partners or altered activities.

In another aspect the invention relates to method for analysing the NER status or genotype of a subject, a tissue sample or cell or cell-line, in particular its TTDA/hTFB5 genotype. Analysing the NER status genotype is herein understood to mean analysing whether or not a genetic defect is present in the sample that underlies a defect in TFIIH stability and/or levels, a defect in DNA transcription, a defect in NER in general and in particular a defect in the TCR subpathway. The method preferably comprises the use of a nucleic acid molecule or probe or antibody as defined above. The method preferably is a method for diagnosing a TTD deficiency. Alternatively, the method for diagnosing a DNA transcription deficiency or NER/TCR deficiency is a method, which comprises the use of an antibody or antibody-fragment as defined above.

In another aspect the invention relates to an expression vector as defined above, wherein the vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol.10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein. The vectors are preferably formulated in a pharmaceutical composition comprising a suitable pharmaceutical carrier as defined below. Methods for preparing administrable compositions are well known in the art and described in more detail in various sources, including, for example, Remington: The Science and Practice of Pharmacy by Alfonso R. Gennaro, published by Lippincott Williams & Wilkins; 20th edition, Jun. 1, 2003.

In a further aspect the invention relates to the use of a vector suitable for gene therapy as defined above in the manufacture of a medicament for the treatment of a NER, TCR of DNA transcription deficiency, in particular a genetic defect in the TTDA gene. Similarly the invention relates to a method for treating a TTDA genetic defect and/or a TFIIH stabilizing deficiency, wherein the method comprising the step of administering to a subject suffering from a TTDA deficiency, a pharmaceutical composition comprising a vector suitable for gene therapy as defined above, in an amount effective to overcome the deficiency.

The invention further relates to a pharmaceutical preparation comprising a polypeptide with TTDA stabilizing and NER, TCR and DNA transcription promoting activity as defined above. The composition preferably comprises a pharmaceutically acceptable carrier. In some methods, the polypeptide with TFIIH stabilizing activity purified from mammalian, insect or microbial cell cultures, from milk of transgenic mammals or other source is administered in purified form together with a pharmaceutical carrier as a pharmaceutical composition. The preferred form depends on the intended mode of administration and therapeutic application. The pharmaceutical carrier can be any compatible, non-toxic substance suitable to deliver the polypeptides to the patient. Sterile water, alcohol, fats, waxes, and inert solids may be used as the carrier. Pharmaceutically acceptable adjuvants, buffering agents, dispersing agents, and the like, may also be incorporated into the pharmaceutical compositions.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance. Methods for preparing administrable compositions are well known in the art and described in more detail in various sources, including, for example in Remington: The Science and Practice of Pharmacy by Alfonso R. Gennaro, published by Lippincott Williams & Wilkins; 20th edition, Jun. 1, 2003.

### DESCRIPTION OF THE FIGURES

FIG. 1.

TTDA (hTFB5) is part of the TFIIH complex.
a. Immunoprecipitation (I.P.) assays. Lane 1: molecular weight marker; lane 2-4: whole cell extracts (WCE) from MRC5SV (wt), TTD-A and TTD-A+p8; lane 5-6 I.P. from TTD-A and TTD-A+p8 cells extracts. Right panel: 18% SDS-PAGE gel on which are loaded 10 μg of proteins from WCE's and the α-p44 I.P. cell extracts from TTD-A and TTD-A+p8. The blot was immuno-stained with α-HA; the arrow shows the immuno-stained band corresponding to the HA-tagged p8. Middle and Left panel: 11% SDS-PAGE gels on which are loaded WCE and the α-HA I.P. cell extracts from TTD-A and TTD-A+p8. The blots were immuno-stained respectively with α-XPB and α-cdk7; the arrows show the corresponding immuno-stained proteins.
b. The polypeptide p8 contained in the highly purified TFIIH from a HAP column, co-purifies with the other nine identified components as shown at the top of the panel (silver staining of an 15% SDS-PAGE and immuno-blot) and with TFIIH transcription and repair activities. Lower panel: immuno-blotting identifies p8 in HAP fractions. A rabbit polyclonal antibody raised against the C-terminal end of p8 was used for the detection of p8 in the HAP fractions containing TFIIH.
c. MALDI-MS identification of p8. Upper panel: P8 identification by MALDI peptide mass fingerprinting. In the table the experimentally determined values as well as the corresponding theoretical masses of p8 tryptic peptides are listed. Mass accuracy (ppM) and amino acidic sequences are also shown for each peptide. Middle panel: Schematic representation of MALDI peptide fingerprinting results. P8 has been identified as CG14037 gene product in NCBI database

FIG. 2.

Correction of the DNA-repair deficiency of TTD-A by hTFB5.
a. Microneedle injection of an expression vector containing the wt cDNA of hTFB5 (p8) in TTD99RO (TTD-A) primary fibroblasts. Micrographs show the effect of p8 expression on DNA repair activity. Injected polynucleated primary fibroblasts (2), indicated by the arrow, exhibit a high (wild-type) level of UV-induced UDS, as apparent from the number of silver grains above their nuclei, when compared to the non-injected surrounding TTD-A fibroblasts (1), which express low residual UDS levels typical for TTD-A. UDS correction by p8 microinjection was also observed when injected into the other TTD group A cells, TTD1BR and TTD13PV (not shown).
b. Table summarizing the mutations found in the four cell lines analyzed derived from three unrelated TTD-A cases. In bold the mutated base in the codon.

FIG. 3.

Nucleotide excision repair characteristics of TTDA.
a. UV-survival using wild-type MRC5SV (closed black diamonds), TTD1BRSV (closed red triangles) and a cell population of TTD1BRSV expressing TTDA-HA (closed green squares). The percentage of surviving cells is plotted against the UV-C dose, in J/m².
b. Locally WV-irradiated (40 J/m²) TTDA-HA expressing TTD1BRSV are stained with α-HA (left panel) and α-XPB (right panel) antibodies. Locally damaged areas are indicated by the arrows.
c. TFIIH HAP fraction 6 (1 μl) was pre-incubated with either no antibody (lane 1), or increasing amounts of Ab-p8 (1, 5 and 10 μg; lanes 2-4 respectively), or Ab control (1, 5 and 10 μg; lanes 5-7 respectively) before being supplemented with all other NER factors; XPC-hR23b, XPA, RPA, XPG and ERCC1-XPF in addition to damaged DNA to measure dual incision.

FIG. 4.

Recovery of reduced concentrations of TFIIH in TTD-A fibroblasts expressing TTDA-HA.
a. Mass population of TTD1BRSV expressing TTDA-HA. Cells expressing TTDA-HA (left panel) show an increased level of XPB protein (right panel).
b. Mass population of TTD1BRSV expressing TTDA-HA. ERCC1 levels (right panel) are not influenced by TTDA-HA expression (left panel).
c. TTDA-HA expressing MRC5SV cells (left panel) show an increased level of XPB protein (right panel).

FIG. 5.

Primary amino acid sequence alignment of human TFB5 with its homologs, showing conserved homology of hTFB5 along evolutionary lines. Alignment. Displayed are TFB5 homologs in H. Sapiens, M. musculus, A. thaliana, the C. elegans (all 71 aa), C. reinhardtii (78 aa), S. pombe (68 aa) and S. cerevisiae homolog (72 aa). Identical amino acids are in dark shaded boxes and physico-chemically related residues are in light shaded boxes.

### EXAMPLES

Isolation and Characterisation of cDNA and Genomic Clones Encoding a Human Orthologue of Yeast TFB5.

### Example 1

### Cloning of hTFB5/TTDA

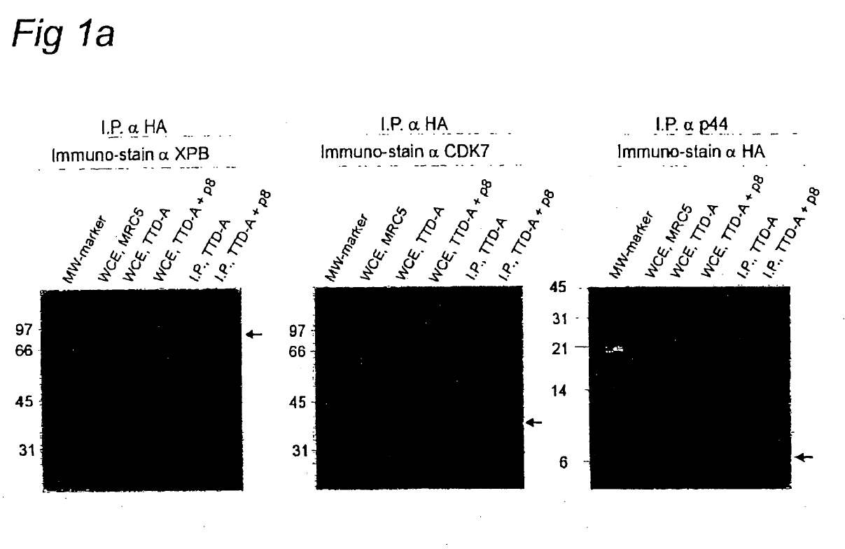

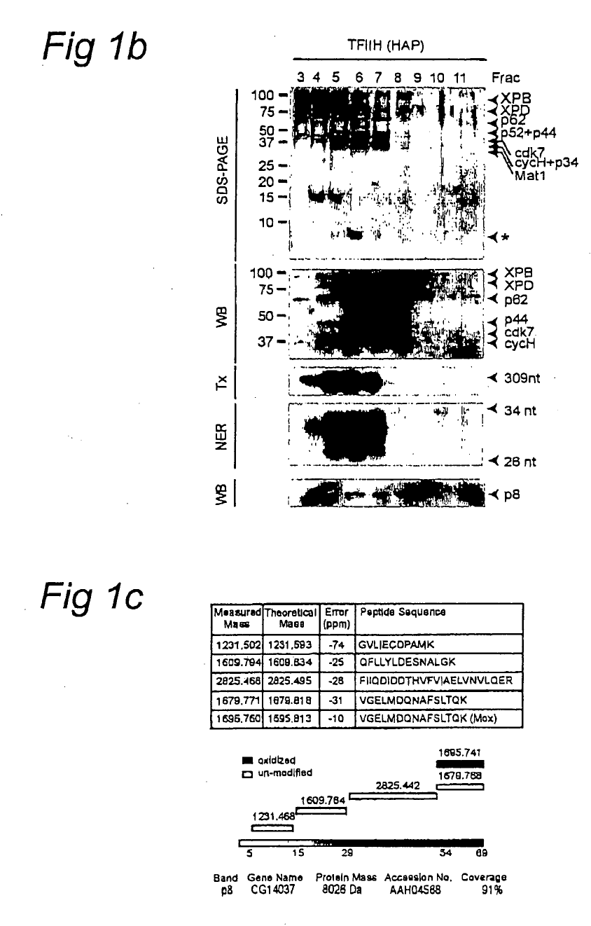

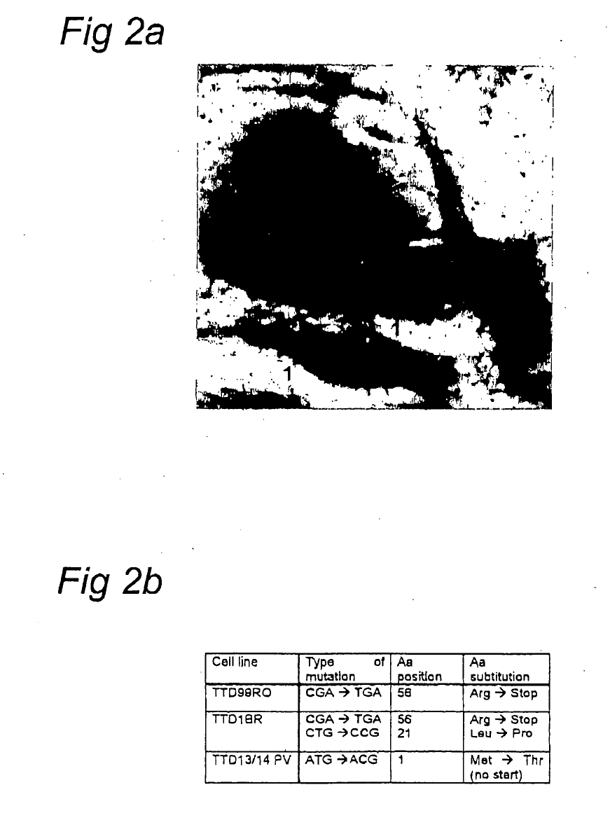

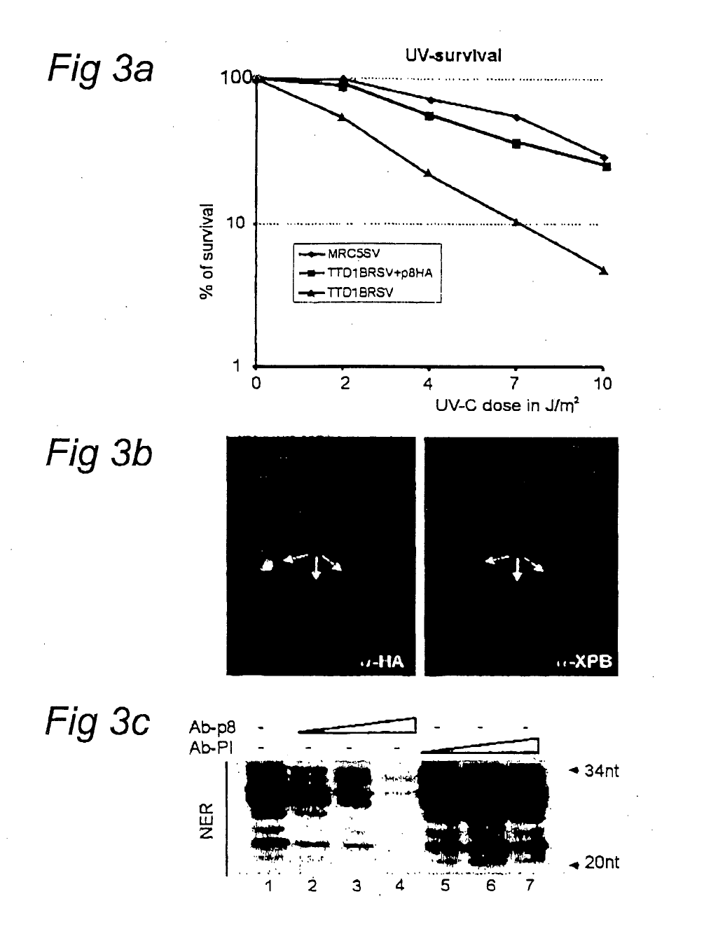

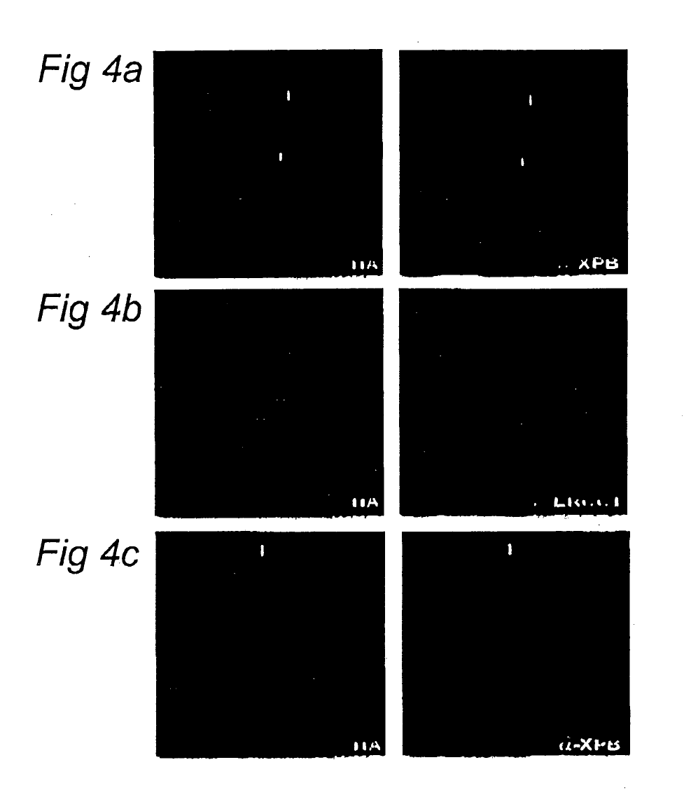

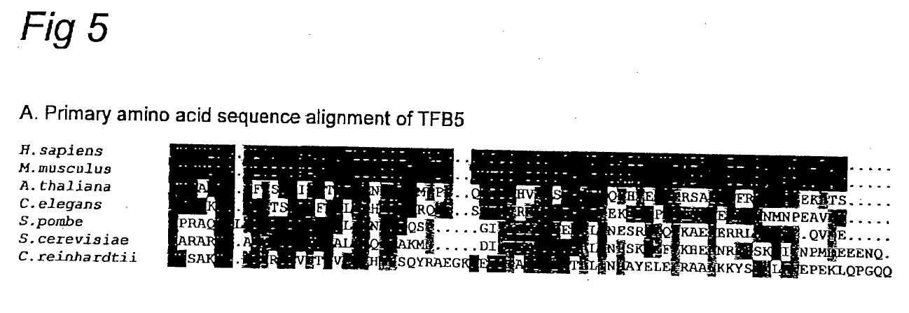

We cloned the human TFB5 cDNA from primary fibroblasts by virtue of its homology with yeast TFB5 using conventional techniques known in the art. An HA-tagged version of hTFB5 (p8-HA) was constructed via conventional molecular cloning methods (Maniatis et al, supra) and expressed in TTD-A fibroblasts and subsequently used for immuno-precipitations with anti-HA (FIG. 1a). Both the XPB core TFIIH and the associated CAK component (CDK7) were co-precipitated with p8-HA, in contrast to replication/repair factor RPA1 (data not shown). Conversely, anti-p44 (another core-TFIIH component) co-precipitated p8-HA. We conclude that, analogous to yeast, this small polypeptide is associated with TFIIH in mammalian cells.

We further analyzed the association of p8 with TFIIH using—purified TFIIH from HeLa cells (Gerard, M. et al., J. Biol. Chem. 266, 20940-20945, 1991). Silver-staining revealed that a protein-band of ˜8 kDa co-purified with the other TFIIH subunits and with the transcription and DNA repair activities (FIG. 1b). MALDI peptide mass fingerprint analysis on tryptic digests of this ˜8 kDa band showed that all identified peptides were part of hTFB5 (FIG. 1c). Finally, immuno-blotting using a polyclonal antibody raised against this 8 kDa polypeptide showed that it co-purifies with TFIIH (FIG. 1B). These experiments unambiguously recognized this polypeptide as the human ortholog of TFB5 and as a genuine, novel tenth component of TFIIH.

To confirm that hTFB5 is the gene affected in TTD-A, we microinjected the cDNA into polynucleated TTD-A fibroblasts and determined the DNA repair capacity of injected cells by measuring WV-induced UDS (unscheduled DNA synthesis). FIG. 2a shows a binucleated p8 injected cell (2) with a clearly increased UDS (elevated grain number) as compared to the low UDS in non-injected neighboring TTD-A cells (1). p8 cDNA corrected the repair defect of TTD-A cells to a comparable level as observed in wild-type cells assayed in parallel, indicating that hTFB5 (p8) is affected in TTD group A.

### Example 2

### TTDA Diagnosis of Subjects

To verify that hTFB5 is implicated in TTD-A we analyzed genomic DNA of three unrelated TTD-A cases (Table 1). All three TTD-A patients showed an inactivating, although distinct, mutation within the hTFB5 gene (FIG. 2b). Patient TTD99RO carries a homozygous C→T transition at codon 56, converting CGA (arginine) into a TGA stop codon, truncating the protein by 23%. Siblings TTD13PV and TTD14PV harbor (homozygously) a mutation in the start codon, converting ATG to ACG. This mutation will result in either a complete loss of protein synthesis or the production of an N-terminal truncated polypeptide (lacking the first and most conserved 15 amino acids (21%)), when a downstream AUG at codon 16 is used. Since only one allele was detected, both mutations can be considered functionally homozygous.

Finally, patient TTD1BR appeared a compound heterozygote: one allele being identical to TTD99RO and the other carrying a T→C transition at codon 21, converting the conserved leucine residue to a proline. Genomic mutations were also identified in isolated mRNA, as revealed by RT-PCR, demonstrating that these alleles are expressed (data not shown). Together our data unambigously identified hTFB5 as the NER-defect causing gene in TTD group A. We designated this novel TFIIH subunit as TTDA (Accession nr. AJ634743).

The TTDA protein and DNA coding sequences provided in the sequence listing, SEQ ID No 1 and SEQ ID No. 2 respectively, provide means to diagnose subjects for the presence of a gene defect in the TTDA gene. Detection of gene defects may be performed by various methods known in the art, in situ or in vitro on specimens from a subject such as but not limited to tissue samples, blood samples, hair samples, faeces, urine, saliva. Various techniques may be applied for mutation analysis of TTDA in a subject, such as but not limited to: DNA sequencing of genomic DNA or cDNA, PCR or Nasba analysis, analysis using Southern blotting, expression analysis using northern or western blotting, DNA microarray or protein microarray analysis, SAGE analysis, EST or SNP analysis.

### Example 3

### TTDA Involved in NER Repair, an Assay for TTDA Activity

To further explore the role of TTDA in NER we generated TTD1BR-SV cells stably expressing HA-tagged TTDA. Exposure of the TTDA-HA expressing cells to UV-C light (FIG. 3a) clearly showed that the cells exhibit a TV-sensitivity level comparable to NER-proficient cells assayed in parallel. Using an assay in which UV-damage is locally inflicted in cell nuclei by irradiation through a filter that contains (5 μm) pores, we demonstrated previously that TFIIH components transiently accumulate at these sites (Mone, M. J. et al., EMBO Rep. 2, 1013-10117, 2001, Volker, M. et al., Mol. Cell 8, 213-224, 2001 and Hoogstraten, D. et al, Mol. Cel. 10, 1163-1174, 2002). We applied the same procedure on TTDA-HA expressing cells to test participation of TTDA in the NER reaction in vivo. At local damaged sites, marked by the accumulation of endogenous XPB, TTDA-HA also accumulated (FIG. 3b). This indicates that TTDA participates in the NER reaction similarly to other NER factors (Rademakers, S. et al., Mol. Cell. Biol. 23, 5755-5767, 2003).

We further developed an assay for testing the involvement of TTDA in NER in a reconstituted in vitro incision/excision repair assay, using recombinant NER factors, purified TFIIH and a plasmid containing a cisplatin-adduct. Addition of increasing amounts of purified anti-TTDA (p8) polyclonal antibodies incubated with a fixed amount of purified TFIIH clearly inhibited the repair reaction, whereas treatment with pre-immune serum had no effect (FIG. 3c). In conclusion, these results demonstrate that TTDA participates, as a part of TFIIH, in NER.

Moreover, a functional assay is here provided which allows testing of TTDA protein preparations and TTDA proteins that are mutated or chemically or post translationally modified, for NER promoting activity. The reconstituted in vitro incision/excision repair assay described above (and as previously described in Shivji et al., 1999, Methods Mol. Biol, 113, 373-392) may be stripped/cleared from (endogenous) TTDA proteins using polyclonal or monoclonal antibodies using conventional techniques, for instance binding to immobilized secondary antibodies or immobilized protein A or protein G. Subsequently TTDA proteins, in particular variants forms thereof as described herein above, from various sources and preparations, chemically synthesized, in vitro translated preparations or purified TTDA from in vivo sources such as micro organisms, plants or animals, may be assayed for their NER promoting activity in the in vitro repair reaction of the reconstitution assay.

### Example 4

### TFIIH Stabilization Assay

The NER defect and TTD-specific features in TTD-A appeared to be linked to a reduced steady-state level of the total amount of TFIIH (Vermeulen, W. et al., Nat. Genet. 26, 307-13, 2000). As shown in FIG. 4a, cells that express TTDA-HA have a concomitant higher XPB level, suggesting that TTDA has a stabilizing function and protects TFIIH from degradation. Similar observations were made in cells stained with antibodies directed against other TFIIH components, i.e. p62, p44 and CDK7, indicating that the intra-nuclear levels of the entire TFIIH complex are increased. In contrast, non-TFIIH NER factors (ERCC1) are not increased (FIG. 4b). Additionally, in immunoblot experiments (FIG. 1a) elevated levels of XPB and CDK7 were also observed after TTDA expression.

These experiments show that the TFIIH concentration depends on the presence of the TTDA protein and hence provide a functional assay for TTDA protein activity. The assay here provided allows functional testing of TTDA from various sources and preparations, TTDA mutants, (both naturally occurring mutants and constructed mutants) and modified proteins. We transiently over-expressed TTDA-HA in MRC5 (wild-type SV40-immortalized). Even in these TTDA-proficient cells transient over expression of the TTDA gene appeared to increase the cellular TFIIH content (FIG. 4c), showing that TTDA expression regulates TFIIH concentration in vivo. Overexpression of (functional) TTDA genes in TTDA-deficient cells enhances the relative TFIIH stabilization effect, whereas dysfunctional TTDA mutants have a diminished effect or no effect, depending on the residual activity of the mutant.

One of the most obvious cellular phenotypes of cultured TTD-A cells is the severely reduced level (˜30%) of TFIIH (Table 1). The fact that TTDA transiently increases the level of TFIIH even in wild-type cells (FIG. 4c) demonstrates that this 8 kDa protein is involved in regulating steady-state levels of TFIIH. Because mRNA levels of TFIIH components are not reduced in TTD-A cells (Vermeulen et al, 2000 supra), controlling the amounts of TFIIH by TTDA must be post-transcriptional. Regulation at the translational or post-translational level suggests a chaperone-like function of TTDA for complex-assembly or maintenance of the complex structure. An altered tertiary structure of TFIIH, by the absence of TTDA, may impede both the longevity of this complex as well as its repair and transcription activity.

Although striking parallels on TFIIH composition, structure and functioning (Table 2) can be drawn between man and yeast, differences in TFB5's relative contribution to transcription efficiency are also observed. In yeast the absence of TFB5 appeared to affect the efficiency of transcription and NER, whereas in man TTDA mutations affect transcription to some extent (Tirode, F., Busso, D., Coin, F. & Egly, J. M., Mol. Cell 3, 87-95, 1999). Furthermore, in man the absence of TTDA appeared to predominantly affect TFIIH stability and or steady-state levels, whereas in yeast, absence of TFB5 did not reduce TFIIH levels.

The severe effect on NER functioning suggests that NER requires higher concentrations of TFIIH than transcription. Recent live cell studies, showed that TFIIH participates significantly longer in NER than in transcription (Hoogstraten, D. et al., Mol. Cel. 10, 1163-1174, 2002), providing a possible explanation for the increased need for sufficient amounts of TFIIH in NER. Additionally, an altered structure of TFIIH by mutated TTDA may mainly affect the NER function.

In conclusion, the identification of TTDA as a novel, evolutionarily conserved, tenth subunit of TFIIH provides essential information to understand the molecular mechanism of TFIIH function in DNA repair, transcription, and human disease. These findings unambiguously identify TTDA as the NER-defect causing gene in TTD group A, and provide an opportunity to diagnose and treat disorders caused by insufficient cellular levels of TFIIH, which may lead to deficiency in pol I and pol II DNA transcription, NER deficiency and in particular TCR deficiency in a subject.

Methods and Materials

Cloning. For molecular cloning techniques general reference is made to Sambrook and Russell, 2001, Molecular Cloning: A Laboratory Manual, 3rd edition, Cold Spring Harbor Laboratory Press, Cold Spring Harbor N.Y., and to Current Protocols in Molecular Biology, F.M. Ausubel ed., published by John Wiley & Sons Inc., December 2003. Nucleic acid modifying enzymes, kits, equipment and automated DNA synthesisers were used according to the manufacturer's instructions.

Cell lines, culture conditions and transfections. Primary human fibroblasts TTD99RO, TTD1BR, TTD13PV, TTD14PV (all TTD-A) and C5RO (wild type, NER-proficient) were cultured under standard conditions at 37°, 5% CO₂ and 3% oxygen in Ham's F-10 medium supplemented with 12% FCS and antibiotics (penicillin and streptomycin). SV-40 transformed cell lines TTD1BR-SV (TTD-A) and MRC5-SV (wild type) were cultured at 37°, 5% CO₂ in a 1:1 mixture of F-10 and DMEM (Gibco) supplemented with 8% FCS and antibiotics. C-terminally HA-tagged TTDA was cloned into the expression vector pCDNA3 (Invitrogen), this construct was transfected into TTD1BRSV fibroblasts using FUGENE 6 transfection reagent (Roche). Transfectants were submitted to triple ultraviolet-C irradiation of 8 J/m2 with two-days intervals to enrich the population of TTDA-HA expressing cells. One week after the last irradiation stable transfectants were single cell sorted (FACSVantage, Becton Dickinson, Belgium). Single cell derived colonies were tested on the expression of the fusion gene by anti-HA immunofluorescence. A selected clone, stably expressing TTDA-HA was tested for its repair capacity (UV-survival, see below).

Immunoprecipitation and immunoblot analysis. Whole cell extracts from MRC5SV, TTD1BRSV and TTD1BRSV+hTFB5HA were prepared by isolating cells from 6 petri dishes per cell line (13 cm). Collected cell-pellets were washed once in PBS prior to lysis by douncing (20 strokes using a 12.61 mm dounce homogenizer, Bellco Glass, INC., USA) in 2 ml of buffer A (50 mM Tris pH 7.9; 150 mM NaCl; 20% Glycerol; 0.1% NP40; 5 mM β-mercaptoethanol) supplemented with anti-proteases. Cellular extracts from MRC5SV, TTD1BRSV and TTD1BRSV+hTFB5HA were incubated overnight at 4° C. in buffer A with respectively anti-HA (12CA5) and anti-p44 (1H5) anti-bodies cross-linked to protein A-sepharose beads (Amersham Biosciences, UK). Prior to immuno-precipitations, cross-linked beads were washed 3 times in buffer A. After incubation with the extracts beads were extensively washed with buffer A, prior to analysis by SDS-PAGE and immunoblotting using anti-XPB (1B3), anti-cdk7 (2F8) and anti-HA (3F10).

Mass Spectrometry.

TFIIH subunits were separated on a 15% polyacrylamide SDS-PAGE and silver stained (Rabilloud, T. Silver staining of 2-D electrophoresis gels. Methods Mol. Biol. 112, 297-305, 1999). The band migrating below the 10 kDa marker was excised and in gel-digested with trypsin (Cavusoglu, N., Brand, M., Tora, L. & Van Dorsselaer, Proteomics 3, 217-223, 2003). Peptide extracts were speed-vac concentrated, chromatography purified on a C18 reverse-phase “ZipTip” pipette tip (Millipore) and finally eluted in 2 μl of acetonitrile 50%. 0.5 μl of peptides were mixed either with an equal volume of saturated alpha-cyano-4 hydroxycinnamic acid (dissolved in 50% acetonitrile) or with a same volume of saturated 2,5-dihydroxybenzoic acid (dissolved in 20% acetonitrile). Both matrices were purchased from LaserBio Labs. Mass measurements were carried out on a Bruker Reflex IV MALDI-TOF spectrometer in the positive-ion reflector mode. The mass acquisition range was 800-3000 Da with a low mass gate set at 700 Da. Internal calibration was performed using autolytic trypsin peptides (MH⁺ with m/z=842.51, 2211.11 and 2807.47). Mono-isotopic peptide masses were assigned manually using the Bruker X-TOF software. Database searches (NCBI, proteins of human origin), using parameters: MW between 0-10 kDa; trypsin digestion (one missed cleavage tolerated), cysteines modified by carbamidomethylation, methionine oxidation and mass tolerance of 75 ppM, were performed with the Profound program (http://prow1.rockefeller.edu/): 4 out of 5 p8 peptides were detected in the MALDI spectrum using both alpha-cyano-4 hydroxycinnamic (data not shown) and 2,5-dihydroxybenzoic acids as matrix

Microneedle injection and DNA repair assays. HTFB5 cDNA constructs (with and without HA-tag) were microinjected into TTD-A fibroblasts (TTD99RO, TTD1BR) as described (Vermeulen, W. et al,. Am J Hum. Genet. 54, 191-200, 1994). Briefly, 3 days prior to microinjection cells were fused using inactivated Sendai virus and seeded onto coverslips. 24 hrs after injection, to allow the expression of the injected gene, cells were exposed to 15 J/m² UV-C and incubated for 2 hr in culture medium supplemented with ³H-thymidine. After removing the excess of free ³H-thymidine, cells were fixed. DNA repair capacity (WV-induced unscheduled DNA repair synthesis or UDS) was determined as the amount of autoradiographic grains above the nuclei, which is usually severely reduced in NER-deficient cells (for details see Vermeulen, W. et al., Nat. Genet. 26, 307-13, 2000).

The sensitivity towards ultraviolet light of the TTD1BRSV (TTD-A) fibroblasts, TTD1BRSV+hTFB5-HA (TTD-A corrected) and the NER-proficient MRC5SV (wt) human fibroblasts was determined by ³H-thymidine incorporation, as described previously (Sijbers, A. M. et al., Nucleic Acids Res. 24, 3370-3380, 1996 and van Gool, A. J. et al., Embo J. 16, 5955-5965, 1997).

Mutation Analysis on hTFB5 Gene and cDNA.

Total RNA (10 μg) was extracted from wild type (C5RO) and TTDA primary fibroblasts (TTD99RO; TTD1BR; TTD13PV and TTD14PV) using Rneasy Mini columns (Qiagen, USA) and used for preparation of cDNA by reverse transcription. Reverse transcription was performed using the SuperScript tm First-Strand Synthesis System for RT-PCR (Invitrogen, USA). Amplification of hTFB5 cDNA was carried out using puReTaq™Ready-To-Go™PCR Beads (Amersham, USA) and 10 pmol of primers (Tm: 60°).

The coding region of the genomic hTFB5 was amplified by using 2 sets of primers, covering the coding exons 2 and 3. Primers sequences are available on request. Genomic DNA (100-500 ng) was amplified by using puReTaq™Ready-To-Go™PCR beads (Amersham, USA) and 10 pmol of each primer set. Amplification products were cloned into T-easy vector (Promega, USA). For each cell line 18 independent clones were sequenced by BaseClear labservices (BaseClear, The Netherlands).

Immunofluorescence. Two days after micro-needle injection or transfection, cells were washed twice in PBS and then fixed in 2% paraformaldehyde and permeabilized by 2 times 10 minutes rinsing in PBS containing 0.1% Triton X-100 (PBS-T), followed by washing with PBS+ (PBS+0.15% glycine and 0.5% BSA). Antibodies, diluted in PBS+ were incubated for 2 h. at RT in a moist chamber, dilutions are: anti-XPB (IB3), 1000×; anti-HA (3F10, Roche), 1000×. Subsequently, coverslips were washed (5×; 5 min) in PBS-T, and incubated with the secondary antibody, respectively: goat anti-mouse, Cy3-conjugated (The Jackson Laboratory); goat anti-rat, Alexa 488-conjugated (Molecular Probes), each diluted 1000× in PBS+. After the same washing procedure, coverslips were mounted in Vectashield mounting medium (Vector Laboratories) containing DAPI (4′-6-diamino-2-phenylindole) at 1.5 μg/μl. Epifluorescent and phase-contrast images were produced on a Leitz Aristoplan microscope equipped with epi-fluorescence optics and a PLANAPO 63×/1.40 oil immersion lens.

Dual incision NER assay. Dual incision assay was carried out as described previously (Riedl, T., Hanaoka, F. & Egly, J. M., Embo J. 22, 5293-5303, 2003)., in 25 μl dual incision buffer supplemented with 2 mM ATP. Each reaction contained 5 ng of XPG, 15 ng of XPF/ERCC1, 10 ng of XPC-hHR23B, 50 ng of RPA, 25 ng of XPA and TFIIH as indicated. Following 10 min of pre-incubation at 30° C., 30 ng of Pt DNA is added and the reactions are continued for 90 min at 30° C. The excised fragment is detected on 14% urea-acrylamide after annealing with 9 ng of the complementary oligonucleotide and addition of four radio labeled dCMPα-P³² (3000 μCi/mmol) residues by Sequenase V2.1 (USB). Prior to the addition to the repair reactions, TFIIH was preincubated with either p8 polyclonal anti-bodies or pre-immune serum. The rabbit polyclonal was produced by coupling the peptide VGELMDQNAFSLTQK (57-71) to ovalbumin and injecting it in rabbit.

Local Ultraviolet Irradiation. Cultured cells were UV-irradiated (254 nm) at 40 J/m² through an isopore polycarbonate filter (Millipore) containing pores of 5 μm in diameter as described previously (Mone, M. J. et al., EMBO Rep. 2, 1013-10117, 2001 and Volker, M. et al., Mol. Cell 8, 213-224, 2001). Subsequently, after filter removal cells were fixed with paraformaldheyde and further processed for immuno-cytochemistry as described above.

Accession numbers. The complete human TTDA cDNA sequence is available under BC060317, AK055106 or AJ634743. The human genomic TTDA sequence may be found in the human chromosome 6q25.3 contig, which is available under Hs 356224 and NT-007422, chr. 6 contig Hs_{—}7579.

TABLE 1
Clinical and cellular features

| | GENERAL DISEASE CHARACTERISTICS | | | | | | PATIENTS IN THIS STUDY | | |
|---|---|---|---|---|---|---|---|---|---|
| | XP | CS | XP/CS⁽¹⁾ | TTD⁽²⁾ | XP/TTD⁽³⁾ | COFS⁽⁴⁾ | TTD1BR⁽⁵⁾ | TTD99RO | TTD13/14PV |
| Gene(s) | XPA-XPG | CSA, CSB | XPB, XPD, | XPD | XPD | XPD, XPG, | TTDA | TTDA | TTDA |
| | | | XPG | (XPB) | | CSB | | | |
| Cutaneous symptoms | | | | | | | | | |
| photosensitive skin | mild/ | mild | moderate/ | mild/ | severe | severe | mild | very mild | mild |
| | severe | | severe | moderate | | | | | |
| skin cancer | mild/ | — | mild to | — | mild | — | — | — | — |
| | severe | | severe | | | | | | |
| brittle low-sulfur hair | − | − | − | + | +/− | − | + | + | + |
| other⁽⁶⁾ | a, b | — | a, b | c, d | b, d | | d | c, d, e | d |
| CS-like symptoms⁽⁷⁾ | − | ++ | + | + | − | +++ | f, g, i, j | f, g, h, i, j, k | g, i, j |
| Cellular features | | | | | | | | | |
| recovery of transcription | low | low | low | low | low | low | low | low | low |
| after UV (TC-NER) | (groups | | | | | | | | |
| | ABDFG), | | | | | | | | |
| | normal | | | | | | | | |
| | (groups | | | | | | | | |
| | CE) | | | | | | | | |
| overall UV sensitivity | 1.5-10× | 2.5-5× | 4-10× | 1.5-5× | 2.5-10× | 10× | 2.1× | 2.7× | 3.5× |
| TFIIH level | 60-100% | normal | 75% | 30-50% | N.D. | N.D. | ˜30% | ˜30% | ˜30% |
| GG-NER activity | 0-70 | normal | 0-40 | 10-45 | 0-30 | <5 | 15-25 | 20 | 10 |
| (UDS, % of normal) | | | | | | | | | |

Table 1: Legend:
⁽¹⁾Patients with combined features of XP and CS.
⁽²⁾Photosensitive TTD.
⁽³⁾combined XP and TTD features; Broughton, 2001.
⁽⁴⁾severe CS features, Graham, 2001.
⁽⁵⁾Stefanini, 1993.
⁽⁶⁾code: a = actinic keratosis; b = freckling; c = collodion baby; d = ichthyosis; e = eczema.
⁽⁷⁾code: f = cataracts; g = developmental delay; h = deafness; i = mental retardation; j = short stature; k = caries

TABLE 2
TFIIH subunits in mammals and yeast:

| | | | predicted | | % identity |
|---|---|---|---|---|---|
| | | | Protein size in | | between |
| subunit | Mammalian | S. Cerevisiae | kDa (human) | Properties | human yeast |
| 1 | XPB | RAD25/SSL2 | 89 | Helicase, 5′-3′ | 49 |
| 2 | XPD | RAD3 | 80 | Helicase, 3′-5 | 51 |
| 3 | GTF2H1 (hTFB1) | TFB1 | 62 | | 21 |
| 4 | GTF2H2 | SSL1 | 44 | Zn-finger | 34 |
| 5 | GTF2H3 (hTFB4) | TFB4 | 34 | Ring-finger | 29 |
| 6 | GTF2H4 (hTFB2) | TFB2 | 52 | | 34 |
| 7 | MAT1 (hTFB3) | TFB3 | 32 | Ring-finger | 31 |
| 8 | CDK7 | KIN28 | 41 | CTD kinase | 41 |
| 9 | Cyclin H | CCL1 | 38 | cyclin motifs | 23 |
| 10* | GTF2H5 (hTFB5/TTDA)* | TFB5* | 8 | | 28 |

1-29. (canceled)

30. A nucleic acid molecule comprising a nucleotide sequence encoding a polypeptide capable of interacting with TFIIH, wherein the nucleotide sequence is selected from the group consisting of:
a) a nucleotide sequence encoding a polypeptide having an amino acids sequence that has at least 50% amino acid sequence identity with SEQ ID No: 1,
b) a nucleotide sequence that has at least 50% nucleotide sequence identity with SEQ ID Nos: 2 or 3,
c) a nucleotide sequence the complementary strand of which hybridises to a nucleotide sequence of (a) or (b), and
d) a nucleotide sequence which differs from the nucleotide sequence of (c) due to the degeneracy of the genetic code.

31. A nucleic acid according to claim 30, wherein the polypeptide has TFIIH stabilizing activity.

32. A nucleic acid according to claim 30, wherein the polypeptide has NER promoting activity.

33. A nucleic acid according to claim 30, wherein the nucleotide sequence encodes a mammalian TFIIH 8 kDa subunit TTDA.

34. A nucleic acid according to claim 32, wherein the nucleotide sequence encodes a human or a murine TTDA or a derivative thereof.

35. A vector comprising a nucleic acid molecule or probe as defined in claim 30.

36. An expression vector comprising a nucleic acid molecule according to claim 30, wherein the nucleotide sequence encoding the polypeptide is operably linked to a promoter capable of directing expression of the coding sequence in host cells for the vector.

37. A nucleic acid probe, wherein the probe comprises at least 10 contiguous nucleotides of a nucleotide sequence that has at least 90% identity with a nucleotide sequence as depicted in SEQ ID No. 2.

38. The nucleic acid probe according to claim 37 immobilized on a solid support.

39. A vector comprising a nucleic acid molecule defined in claim 30.

40. A vector comprising a probe as defined in claim 39.

41. An expression vector comprising a nucleic acid molecule according to claim 30, wherein the nucleotide sequence encoding the polypeptide is operably linked to a promoter capable of directing expression of the coding sequence in host cells for the vector.

42. The expression vector according to claim 41, wherein the nucleotide sequence encoding the polypeptide comprises a second nucleotide sequence encoding a molecular tag, whereby the second coding sequence is fused in frame to the sequence encoding the polypeptide.

43. The expression vector according to claim 42, wherein the molecular tag is a fluorescent tag or an affinity tag.

44. A host cell comprising a vector according to claim 39.

45. A method for producing a polypeptide with TFIIH interacting or stabilizing activity, the method comprising the step of culturing a host cell as defined in claim 44 under conditions conducive to the expression of the polypeptide, and, optionally recovering the polypeptide.

46. A transgenic animal comprising in its somatic and germ cells a vector according to claim 40.

47. The transgenic animal according to claim 46, wherein the vector is integrated via homologous recombination in the endogenous TTDA locus.

48. The transgenic animal according claim 46, wherein the nucleotide sequence encoding the polypeptide comprises a mutation.

49. The transgenic animal according to claim 48, wherein the mutation is an inactivating mutation.

50. A polypeptide with TFIIH interacting activity and comprising an amino acid sequence having at least 50% identity with the amino acid sequence of SEQ ID No. 1.

51. A polypeptide according to claim 50, wherein the polypeptide has TFIIH stabilizing activity.

52. A polypeptide according to claim 50, wherein the polypeptide has NER promoting activity.

53. An antibody or antibody-fragment that specifically binds to a polypeptide as defined in claim 50.

54. A method for analysing the TTDA genotype of a subject, the method comprising administering a nucleic acid molecule as defined in claim 30, wherein the method is a method for diagnosing an NER deficiency.

55. A method for analysing the TTDA genotype of a subject, the method comprising administering a probe as defined in claim 39, wherein the method is a method for diagnosing an NER deficiency.

56. A method for diagnosing an NER deficiency, comprising administering an antibody or antibody-fragment as defined in claim 54.

57. A method for diagnosing an NER deficiency, comprising administering an antibody or antibody-fragment as defined in claim 55.

58. An expression vector according to claim 39, wherein the vector is a vector that is suitable for gene therapy.

59. A pharmaceutical preparation comprising a vector as defined in claim 58.

60. A method for treating a genetic disorder, comprising administering a medicament comprised of a nucleic acid molecule as defined in claim 30.

61. A method for treating a genetic disorder, comprising administering a medicament comprised of an expression vector as defined in claim 58.

62. A method of producing a medicament for the treatment of a UV sensitivity syndrome or an NER deficiency, comprising administering a nucleic acid molecule as defined in claim 30.

63. A method of producing a medicament for the treatment of a UV sensitivity syndrome or an NER deficiency, comprising administering an expression vector as defined in claim 58.

64. A method for treating a UV sensitivity disorder in a subject, comprising administering a source of a polypeptide as defined in claim 52.

65. The method according to claim 64, wherein the source of the polypeptide is a vector as defined in claim 30.

66. The method according to claim 64, wherein the source of the polypeptide is a vector that is suitable for gene therapy.


SEQUENCE LISTING (5 sequences)

SEQ ID NO: 1 - protein, 71 aa, Homo sapiens
MVNVLKGVLIECDPAMKQFLLYLDESNALGKKFIIQDIDDTHVFVIAELVNVLQERVGELMDQNAFSLTQK

SEQ ID NO: 2 - DNA, 331 bp, Homo sapiens
ggcgcttctgcatctgtgggccgagcattcttcaggtcatctgaaccttctgagaaaacatggtcaacgtcttgaaaggagtgcttatagaatgtgatcctgccatgaagcagtttctgctgtacttggatgagtccaatgccctggggaagaagttcatcattcaagacattgatgacactcacgtctttgtaatagcagaattggttaatgtcctccaggagcgagtgggtgaattaatggaccaaaatgctttttcccttacccagaaatgaaaatactcaatatggaccatttaggaattataagcagcaactgtgaaagacttgcc

SEQ ID NO: 3 - DNA, 89 bp, Homo sapiens
  misc_feature: exon 1, non coding, nucl. 22 - 65
gatggcgtatcggcgaggcgcactctgccggcaacgccgaggcgcttctgcatctgtgggccgaggtgtgtggcgagcgtccgagcggc

SEQ ID NO: 4 - DNA, 110 bp, Homo sapiens
  misc_feature: exon 2 ranges from nucl. 19 to 89 CDS starts at nucl. 55, ATG.
taccttactctttttattagcattcttcaggtcatctgaaccttctgagaaaacatggtcaacgtcttgaaaggagtgcttatagaatggttagtagttttgatactgca

SEQ ID NO: 5 - DNA, 1226 bp, Homo sapiens
  misc_feature: exon 3 ranges from nucl. 27 to 1226 CDS ends at nucl. 206, TGA stopcodon
cttacaatcatgtgtttgtctttacagtgatcctgccatgaagcagtttctgctgtacttggatgagtccaatgccctggggaagaagttcatcattcaagacattgatgacactcacgtctttgtaatagcagaattggttaatgtcctccaggagcgagtgggtgaattaatggaccaaaatgctttttcccttacccagaaatgaaaatactcaatatggaccatttaggaattataagcagcaactgtgaaagacttgccactcaatatcttaggtgactgattagacatagagggttgttttaggagcatgccacgggaaagactgagggatcatgatcatttgttcagaaaaaaagcccctgaactgattttgttaccatagaatttaaaaaaaaaaaaagctttaacagttggctgtaatttggcttttattatcctttattaaaatacaaatgtcaatgcttttcctgcctttttaataccatgtcagtgtaacataggtatttattttgctcatccctgtgatctgtgcatttttgctgcgtggatgtgattgtttggtttcagttagaaacgtcatagatttgctgtttgaatatgccaaggtggggacttagacattatgtacgtctcacaaatcctacctgcataccagtcagctctattgaggaagacaatgtaaaactaatgtaaactacagtttgcatttccctgaaaacagaatattgtttttaagagggttagaaacaaccagtgggaaaggcacatgctgctttgtttagtttttccttgttcaaactttgttggtcacattttcccatctgtattcttttttattaagaaaacagcttagggaggctgaggtgggaggattgcttgagcccaggagtccgaggttgctacaaaaacaccattgcactctggcctggacaacagagagaccctgcctcaaaaaaaaaaaaaaaaaaaaaaaaaattctacttgtaccttattccctatgagaatacttatcaaacttatctaaaaaagaaaaataggaacagccatatgcaaagtcagcccaacaggaaaggacatattagattaaaatacattgcggcctgggcttagtggctcacacctgtaatcccagcactttgggaggctgaggcaggcagatcgcttgagtccaggagtcgaaaccctgtctctacaaaaaaaaaaatacaaaaatctgcct